(19) United States
(12) Patent Application Publication
(10) Pub. No.: US 2008/0096229 A1
(43) Pub. Date: Apr. 24, 2008

(54) Modified Flagellins And Flagellar Filaments Useful As Receptors And Methods For The Preparation Thereof

(76) Inventors: Ferenc Vonderviszt, Veszprem (HU); Peter Zavodszky, Budapest (HU); Szilard Kamondi, Budapest (HU); Istvan Barsony, Budapest (HU)

Correspondence Address: HAHN & VOIGHT PLLC, 1012 14TH STREET, NW, SUITE 620, WASHINGTON, DC, 20005, US

(21) Appl. No.: 11/813,038

(22) PCT Filed: Dec. 29, 2005
(86) PCT No.: PCT/HU05/00142

§ 371 Date: Jun. 28, 2007

(30) Foreign Application Priority Data
Dec. 29, 2004 (HU) P0402683

(51) Int. Cl. C07K 16/00; G01N 33/53

(52) U.S. Cl. 435/7.8; 530/402

(57) ABSTRACT

In general, the invention relates to receptors capable of the efficient recognition and binding of target molecules, said receptors being composed of flagellin proteins which comprise the flagellar filaments of bacteria. More closely, the invention relates to modified flagellins or flagellin fragments useful as receptors, and filamentous receptor-structures comprising them. Moreover, the invention relates to procedures for their preparation and their uses. Flagellin-based receptors can be produced easily and inexpensively by bacteria, and purified with an ease without lysing the cells. Moreover, flagellins, due to their polymerization ability, can be used to build various filamentous structures. Starting from flagellin receptors of the invention filaments of desired length and construction can be prepared with a very high binding site density on their surface. These supramolecular objects may serve as basic recognition units for biological sensors and diagnostic kits.

The invention relates to the preparation of receptors constructed from the flagellin protein, the building block of bacterial flagellar filaments. The receptors of the invention are capable of efficient recognition and binding of target molecules.

Flagellin-based receptors can be produced easily and inexpensively by bacteria, and purified with an ease without lysing the cells. Moreover, flagellins, due to their polymerization ability, can be used to build various filamentous structures. These filamentous structures are stable, resistant to proteases, and can be preserved for a long period of time. Starting from a certain type of flagellin receptors, filaments of desired length can be prepared with a very high binding site density on their surface. Additionally, filamentous receptor structures can be polymerized from differently modified flagellins. These block copolymers can recognize and bind different target molecules in different regions, or they can be immobilized through a certain region (e.g. via their specifically modified subunits), while their other parts are involved in molecular recognition. These supramolecular objects may serve as basic recognition units for biological sensors and diagnostic kits.

### BACKGROUND ART

Flagella are the locomotive organelles of bacteria [Macnab, R. M. (1995)]. The helical filaments of bacterial flagella extend over the surface of the cell membrane (FIG. 1) and are made of several tens of thousands copies of the flagellin protein [Namba, K. and Vonderviszt, F. (1997)]. Flagellar filaments are self-assembling systems, i.e. under suitable conditions flagellin monomers can spontaneously assemble into filaments with a structure identical to that of the native filaments. Polymerization of flagellin subunits can be controlled easily [Asakura, S. (1970); Asakura, S. et al., (1964)], and the obtained filaments are resistant to physical and chemical effects, resistant to proteases and their structure is well known at the atomic level.

The flagellin of the bacterium Salmonella typhimurium consists of 494 amino acid residues [Smith, N. & Selander, R. K. (1990)]. Comparison of the amino acid sequences revealed a high degree of homology in the terminal regions containing about 180 N-terminal and 100 C-terminal amino acids, whereas the central segments are highly variable [Joys, T. M. (1985); Wei, L. N. & Joys, T. M. (1985)]. The molecular mass of flagellins of different origin varies in a wide range (28-65 kDa) and the differences are due to the various sizes of the central region.

By now, the amino acid sequences of flagellins as well as the sequences of the corresponding fliC genes, have been determined in several species. The highly conserved terminal regions and the central variable region can be easily distinguished in the newly published sequences by comparing them with the well-known flagellin sequences, [e.g. Schoenhals, G, and Whitefield, C., (1993)].

In the course of exploring the structure of the flagellar filaments it has become obvious that the flagellin subunits arrange themselves into 11 protofilaments [Namba, K. and Vonderviszt, F. (1997); Namba, K. et al., (1989)]. These protofilaments in strong interaction with each other form the structure of the filaments (FIG. 2). Our previous studies revealed that only the conserved terminal regions of flagellin subunits are involved in filament formation [Vonderviszt, F. et al. (1991); Mimori-Kiyosue, Y., et al. (1997)] while the central region forms the D3 domain exposed on the filament surface (FIG. 3a). This domain is not in contact with the adjacent subunits [Samatey, F. A. et al. (2001); Yonekura, K. et al. (2003)] and has no role in the construction of the filament structure (FIG. 3b). The D3 domain is a good target for genetic engineering studies since it can be easily modified without disturbing self-assembling ability.

The D3 domain is constituted by the 190-284 segment of the amino-acid sequence of the Salmonella typhimurium flagellin. The D3 is an unusual domain of β-sheet structure, consisting of four β-chains and a short α helical segment [Samatey, F. A. et al. (2001); Yonekura, K. et al. (2003)]. The outer surface of the domain—which faces the external medium and which is located farthermost from the axis of the filament—is formed by three loop regions: segments 205-213 (H1), 236-244 (H2) and 261-270 (H3) (FIG. 4). The surface characteristics of the D3 domain can be modified by altering the amino-acid sequences of the loop regions. This allows for the construction of specific binding sites (charge patterns, topography).

In proteomics, medical diagnostics or in the production of pharmaceutical active ingredients as well as in environmental analytics and food safety control, it is a frequent task to reliably detect the individual components of complex mixtures. An extensive research is going on aiming at the utilization of protein-based biosensors or protein chips as detector-units in analytical procedures.

Proteins are capable of highly specific recognition of molecules, whereby they play a significant part both in the communication between the living organism and the environment and in the protection against foreign materials. For example, the function of the immune system is based on the fact that antibodies, such as the immuoglobulin G (IgG) molecules are capable of reliably recognizing millions of types of foreign materials. Nature, actually, generates a few million IgG variants which essentially differ only in the structure of their binding region [Metzger, H. (1990)]. Experience shows that among the numerous variants there are at least a few ones that can specifically bind to a surface region of the foreign material (such as a protein molecule) which has got into the body.

In conventional protein-based biosensors and protein chips, antibodies or their suitable fragments are frequently applied as sensory units [Zhu, H. and Snyder, M. (2003)]; however, their production is expensive, the preparation process is complicated, their stability is often insufficient and they can easily lose their native conformation when bound to the carrier surface. Therefore, it is desirable to replace the antibodies with other protein-based receptors. Applying the principles observed with IgG domains, successful attempts have been made to construct binding sites specific to a given target molecule on proteins that originally have no receptor characteristics [Xu, L. et al. (2002); Skerra, A. (2001); Nygren P. and Uhlen, M. (1997)]. By way of generating numerous mutants by varying the amino acid sequence of the surface loop regions of the domain of fibronectin type 3 and by applying directed evolution techniques Xu et al. produced receptors that could specifically bind the TNF-α protein b [Xu, L. et al. (2002)]. Similarly, Skerra et al. constructed different kinds of receptors from the lipocalin protein [Skerra, A. (2001)].

However, production of protein-based receptors is still expensive and labourous. It would be desirable to create a protein-based receptor family providing a wide range of variations, which could be produced easier and cheaper than earlier receptors. It would be particularly advantageous to produce receptors capable of building various supramolecular objects.

Upon seeking solution to this problem, the present inventors discovered that receptors and filamentous receptor structures can be created from the flagellin protein, the building block of bacterial flagellar filaments. According to our present knowledge, isolated flagellin-based receptor structures have not been manufactured so far.

The practical utilization of recombinant, modified flagellins has been seen mainly in the production of vaccines.

Wu et al. [Wu, Y. J. et al., (1989)], for example, inserted surface epitopes of the hepatitis B virus into the hypervariable region of the gene encoding the Salmonella flagellin, and this construct was expressed in the Salmonella dublin vaccine strain, then animals were immunized with the produced bacteria.

The EP 0419 513 patent [Marjarian et al.] describes certain recombinant vaccines containing fusion flagellin proteins where antigen determinant regions of infectious viruses were inserted into the D3 domain of flagellin and the obtained fusion proteins were expressed in flagellin-deficient bacteria. The bacteria recovered their motility by forming flagella from the recombinant flagellins and proved to be immunogenic. Thus, antibodies could bind the flagellins.

The WO 9534664 application [McCoy et al.] describes Flagellata bacteria expressing and carrying recombinant flagella which contain a fused thioredoxin molecule on their external surface. The authors inserted different peptides into the active local loop of thioredoxin, and the constructs can be used for the preparation of a library. As has been pointed out by the authors, the method is suitable e.g. for epitope mapping or for the identification of different target sequences that can be bound to certain receptors.

In her review Westerlund-Wikström, B. [(2000), p. 224, lanes 10-17] has summarized the applicability of flagellar display technology for displaying antigen epitopes as vaccines, for the analysis of interactions between bacterial adhesion peptides and their target molecules, and for displaying short randomized peptides in fundamental research.

Thus, according to the state of the art, so far it is merely the binding of recombinant bacterial flagella to either antibodies or receptors that has been studied. These studies primarily aimed at vaccine production and the flagella were almost exclusively present in their cell-bound form. Furthermore, several display techniques have been developed for this purpose.

To the present knowledge of the inventors no-one has ever realized so far that by creating a suitable binding region on the flagellin, it is possible to use the flagellins themselves or their properly selected fragments (e.g. their D3 domains) as receptors capable of recognizing and binding target molecules. These flagellins can be used as receptors in their isolated form. Furthermore, it has also been unknown that from the modified flagellin subunits isolated, e.g. reconstituted, flagellar filaments, useful as receptors, can be constructed, provided that the polymerization ability of the subunits is preserved. The suitably tailored filamentous structures either can be used in themselves or a multiplicity thereof, if bound to a carrier, may be applied as nanochips or protein chips. Particularly preferably, it is possible to create sites on the filaments themselves, which allow detection of the molecules which bind to the receptor filaments.

Production of flagellin receptors is simpler and cheaper than that of antibodies or any other well-known protein receptors, and this can be done without lysing the bacterial cells.

Therefore, the invention concerns not a different type of display technology but the application of modified flagellins and flagellar filaments as receptors. In other words, the object of the present invention is not the use of flagellins and flagellar filaments as tools for presenting other proteins or peptides or for studying their interaction; instead, the inventors aimed at modifying the flagellin itself so that it may effectively recognize and bind a target molecule, while its polymerization ability is maintained.

### A BRIEF DESCRIPTION OF THE INVENTION

According to an embodiment, the invention relates to the use of a flagellin as a receptor said flagellin being isolated and modified in its variable regions,

comprising
contacting the studied target substance with the modified flagellin under conditions allowing the binding to the flagellin,
directly detecting the binding of the target molecule to the modified flagellin,

wherein
the modified flagellin is modified in its variable region relative to the variable region of the wild type flagellin, to comprise a binding site capable of recognizing and binding the target molecule, and
at least the terminal regions of the flagellin, responsible for the polymerization, are unmodified or modified only to an extent that the said terminal regions preserve their polymerization ability.

Furthermore, the invention relates to the use of an isolated, preferably reconstituted flagellar filament as a receptor, said filament comprising at least one isolated flagellin, preferably a multiplicity of flagellins modified as described above,

said use comprising,
contacting the studied target substance with the isolated flagellar filament under conditions allowing the binding to the modified flagellin,
detecting the binding of the target molecule to the modified flagellin subunits of the isolated flagellar filament.

According to preferred embodiments, the binding of the target molecule is detected by a method selected from the following methods:
a microscopical method, e.g. electron microscopy, preferably, following the attachment of the target molecules to nanospheres,
by reporter molecules (e.g. molecules carrying cromophore groups, preferably fluorescent molecules) placed close to the binding sites, where the spectroscopical property of the reporter molecule changes upon binding,
by immobilizing the target molecule to the surface of a sensor chip (or cantilever oscillator), where the detection is preferably carried out by surface plasmon resonance spectroscopy, by micro-electromechanical sensor, or by e.g. optical waveguide sensors.

In another preferred embodiment of the use of the invention target molecules are obtained from or detected in a sample (e.g. biological or other sample) by using the receptors of the invention. Preferably, the receptors are bound to a carrier, which can be a carrier of any suitable surface, such as glass or quartz, silicium plate, or even a chromatographical matrix. In the use of the invention receptors can be developed for any target molecule. According to certain embodiments of the invention, the target molecule is a molecule different from protein molecules. Preferably, the target molecule differs from immunoglobulins or immunoglobulin fragments and/or from receptor proteins.

The invention further relates to any of the above uses, wherein the D3 domain of the flagellin is modified, preferably the hypervariable regions and/or the surface loop regions of the D3 domain are modified.

The invention further relates to any of the above uses, wherein the terminal regions and the D2 domain of the flagellin are unmodified or modified only to such an extent that the produced flagellin derivatives preserve their polymerization ability.

According to another aspect, the invention relates to a flagellin or flagellin fragment that can be used as a receptor, said flagellin or flagellin fragment containing a modified D3 domain compared with the D3 domain of a wild type flagellin of a given bacterium species or strain, wherein the D3 domain contains one or more modified peptide segment(s) said segment(s) being inserted into one or more hypervariable region(s) and/or surface loop region(s) of the D3 domain and/or the peptide segment(s) replace(s) one or more native segment(s), and the peptide segment or peptide segments together constitute a binding site capable of recognizing and binding the target molecule.

Preferably, the D3 domain of the modified flagellin or flagellin fragment is modified only to an extent that, with the exception of the modified segment(s) and their local environment, the native fold characteristic to the wild type flagellin is essentially retained.

Further preferred flagellins or flagellin fragments are e.g. those, in which
a larger peptide segment is replaced in the hypervariable region and/or surface loop region of the D3 domain, or
the inserted or replaced peptide segment is smaller than a complete protein or protein domain having a tertiary structure of its own, or
the inserted or replaced peptide segment is a complete binding protein or binding domain having a tertiary structure of its own and carrying a binding site of its own.

Optionally, the inserted peptide segment is not a fusion protein or fusion domain, preferably, is not a thioredoxine-like protein or thioredoxin.

In a preferred variation, the peptide segment inserted into the surface loop region of the D3 domain is not longer than the loop region or than 4-times, preferably 3-times, 2-times, 1.2-times, highly preferably 1.2-times the length of the replaced original peptide segment.

According to a preferred embodiment, the flagellin or flagellin fragment modified in the D3 domain is capable of polymerizing into flagellar filament, preferably its terminal regions responsible for the polymerization are unmodified or modified only to an extent that they preserve their polymerization ability.

According to an alternative embodiment, the terminal regions of the flagellin fragment useful as receptors are partly or completely missing. Preferably, such a flagellin fragment comprises only a modified D3 domain as a region essentially originating from flagellin.

According to a further aspect, the invention relates to a flagellin-based polypeptide library, wherein the individual flagellin-like polypeptides contain at least the following:
a) peptide segment(s modified relative to a peptide segment of a wild type flagellin sequence, said segments being inserted into a variable region of a flagellin typical of a given bacterial species, strain or variant, or replacing this variable region partly or completely,
b) non-variable flagellin region(s),

wherein, in the library the segments according to a) show significant variability, while regions according to b) do not show significant variability.

Preferably, in the library the segments specified in a) differ from each other, while the segments defined in b) do not show significant differences, e.g. their average identity with each other is at least 50%, preferably, at least 70%, 75%, 80%, 85%, 90%, 95% or at least 98%, or they are identical even in 100%, or their characteristic parts—such as their polymerization-determining regions, e.g. their terminal regions—are identical or differ only to such an extent that they preserve the function typical of the given flagellin. This function is preferably the polymerization ability of the given flagellin.

In the peptide library of a preferred embodiment, the polypeptides comprise the terminal region(s) typical of the flagellin. Preferably, the modified peptide segment is present in the D3 domain of the variable region of the polypeptide, more preferably at least one of the hypervariable regions and/or surface loop regions within the D3 domain is modified, and, preferably, the modification is located within any of the said regions.

The altered peptide segments in a preferred polypeptide library of the invention are at least partly modified, optionally, most of them are modified randomly.

According to another aspect, the invention relates to a polypeptide library based on the D3 domain of flagellins, where the individual polypeptides contain at least the following:
a) peptide segment(s modified relative to a peptide segment of a wild type sequence, said segments being involved in the hypervariable regions and/or surface loop regions of the flagellin D3 domain typical of a given bacterial species, strain or variant, or replacing this region partly or completely by peptide segment(s), that was/were modified relative to the peptide segment characteristic of the wild type
b) other regions of the flagellin D3 domain typical of the species, strain or variant, wherein, in the said library the segments according to a) show significant variability, while regions according to b) do not show significant variability.

Preferably, in the library the segments defined in a) differ from each other, while the segments defined in b) show minor differences, e.g. their average identity with each other is at least 30%, preferably at least 40%, 50%, 60%, 70%, 80%, 90%, 95% or at least 98%, or they are identical up to 100%.

Preferably, the altered peptide segments are at least partly randomly modified, or optionally their major part is randomly modified.

According to another aspect, the invention relates to polynucleotide libraries, wherein the individual polynucleotides contain sequences encoding the modified flagellins or flagellin fragments of any of the polypeptide libraries of the invention.

According to a further aspect, the invention relates to a combined library comprising any of the polypeptide libraries of the invention and the polynucleotide library comprising the polynucleotide sequences encoding the modified flagellins or flagellin fragments of the said polypeptide library, wherein any modified flagellin or flagellin fragment is present in association with one or more polynucleotide(s) encoding it, thereby allowing for the selection of a polynucleotide encoding the polypeptide capable of binding a given target molecule.

The combined library of the invention is preferably:
a bacterial library
a yeast library
a phage-display library
a yeast double hybrid system
a ribosome-display library
a mRNA-protein fusion library, or
a protein fragment complementation library.

According to a further aspect, the invention relates to a directed evolution method for the production of modified flagellins or flagellin fragments capable of recognizing and binding a given target substance, wherein
i) a combined library according to the invention is prepared,
ii) the library is contacted with the target substance,
iii) polynucleotide elements of the library are selected, wherein the polypeptides belonging to said polynucleotides are capable of binding the target substance,
iv) given regions of the selected polynucleotides are subjected to mutagenesis, preferably to limited random mutagenesis or random mutagenesis,
v) polypeptides are obtained from these modified polynucleotides, and
vi) steps ii)-v) steps are repeated until the polypeptide is capable of binding the target substance in a desired degree, whereby a flagellin or flagellin fragment usable as receptor is obtained.

According to a preferred embodiment, a starting library is prepared by choosing the peptide segment to be altered from wild type flagellin or flagellin fragment typical of a given bacterial species, strain or variant, then the peptide segment(s) is/are subjected to random mutagenesis to produce modified peptide segment(s).

According to a further preferred embodiment, the starting library is prepared by choosing the peptide segment to be altered from wild type flagellin or flagellin fragment typical of a given bacterial species, strain or variant, then in each polypeptide the peptide segment is replaced with another peptide segment or a peptide segment inserted into the peptide segment to be altered by using directed mutagenesis.

According to another embodiment, the starting library is established by combining methods of the two above embodiments.

Preferably, after step vi), steps ii)-v) are repeated by subjecting a polynucleotide region, chosen in a different way, to mutagenesis until a polypeptide capable of binding the target substance to a desired extent is obtained, and thereby a flagellin or flagellin fragment useful as receptor is produced.

According to a further embodiment, the invention relates to a flagellin or flagellin fragment useful as receptor and which can be produced by directed evolution, and

which comprises one or more modified peptide segments forming a binding site capable of recognizing and binding the target substance. The peptide segments are modified relative to the wild type flagellin or flagellin fragment typical of a given bacterial species, strain or variant.

The properties of the preferred flagellin receptors and fragments are listed above.

A further flagellin or flagellin fragment that can be preferably used as a receptor is immobilized to a carrier.

The invention further relates to a protein chip containing several different flagellins or flagellin fragments of the invention, which can be used as receptors, and in which the flagellins or flagellin fragments are immobilized on a carrier, preferably in a matrix arrangement.

Furthermore, the invention relates to the use of any of the above flagellin fragments of the invention as receptors,

whereby
the target substance is contacted with the flagellin fragments under conditions allowing binding to take place,
the binding of the target substance to the modified flagellin fragment is detected.

Preferably, the flagellin fragment does not contain the terminal regions of the flagellin, more preferably, it contains essentially the variable regions, highly preferably, it consists of a modified D3 domain.

According to a farther aspect, the invention relates to a filamentous receptor structure that comprises an isolated flagellar filament consisting of flagellin subunits, and in which at least one of the flagellin subunits is an artificially modified receptor subunit, in which
a variable region is modified relative to the variable region of the wild type flagellin, and comprises a binding site capable of recognizing and binding the target substance, and
the terminal regions responsible for polymerization are unmodified, or modified only to such an extent that they preserve their polymerization ability.

Preferably, the invention relates to filamentous receptor structures comprised of reconstituted flagellar filaments.

Preferably, the filamentous receptor structure of the invention contains artificially modified receptor subunits modified in the D3 domain of the variable region. A binding site capable of recognizing and binding the target substance is created on the D3 domain preferably by altering at least one of the hypervariable regions and/or surface loop regions. More preferably, the alteration is present within any of the above.

According to a preferred embodiment, the filamentous receptor structure comprises, as a receptor subunit, one or more flagellins selected from the following:
a flagellin of the invention as described above, which can be used as a receptor, or a flagellin produced by the directed evolution method of the invention,
a flagellin containing a binding segment inserted by directed evolution, preferably a fusion protein,
a flagellin containing a molecule or moiety carrying a binding site and conjugated with the D3 domain, where the molecule or the moiety is preferably any of the following: a complexing group, e.g. a group capable of binding ions, etc., a protein or protein domain having receptor properties and/or a binding site or binding domain of a protein, etc.

According to a preferred embodiment, the complexing group is a group capable of binding ions, preferably having a calcium, magnesium, iron, nickel, cobalt, manganese, zinc, or arsenic ion binding site. Moreover, a more or less specific binding site can be formed, which is capable of binding several kinds of ions, such as heavy metal ions.

According to a farther preferred embodiment, a domain or prosthetic group (such as hem or chelating group) capable of binding a metal ion can be attached to the surface of the D3 domain, thereby providing the formation of the appropriate binding site.

According to a preferred embodiment, the filamentous receptor structure comprises a conjugating subunit, which is a flagellin carrying or comprising a group capable of the covalent binding of a conjugate, preferably; a flagellin containing cysteine side-chain in its D3 domain, more preferably, in any of the surface loop regions.

Alternatively, the conjugating subunit is also a receptor subunit.

According to a preferred embodiment, the filamentous receptor structure also comprises a detecting subunit that is capable of changing at least one of its detectable properties as a result of one or more binding events.

According to various embodiments,
the detecting subunit comprises a detecting group containing or carrying, preferably
   a chromophore, preferably fluorescent molecule, protein or group, or
   carries metal ions and/or
the detecting subunit is located sufficiently near the receptor subunit to have any of the properties of the detecting subunit detectably changed as a result of binding to the receptor subunit. The detecting subunit may be, for example, located in an adjacent subunit or the detecting subunit may function as a receptor subunit at the same time.

According to a further embodiment,
the molecule or molecule portion carrying the binding site and conjugated to the D3 domain is attached through the cysteine side chain and/or
the detecting group is attached through the cysteine side chain.

According to an embodiment, essentially all of the flagellin subunits are uniform in the filamentous receptor structure of the invention.

According to an alternative embodiment, the filamentous receptor structure comprises at least two types of flagellin subunits, preferably it contains
a) at least two types of receptor subunits, and/or
b) at least one type of receptor subunit and a subunit with a structure similar to that of the wild type subunit, and/or
c) a receptor subunit and, separately, a detecting subunit, and/or
d) any combination of a), b) and c).

According to an embodiment, more than one types of subunits are contained in the filamentous receptor structure, which comprises these subunits in a randomly polymerized form. According to a further preferred embodiment, the different types of flagellin subunits are located in different regions—preferably, in blocks positioned essentially perpendicular to the longitudinal axis—along the filament. Preferably, the bands formed by the receptor subunits and/or the detection subunits are separated from each other by other subunits, e.g. subunits with a structure similar or identical to the wild type structure, as spacer bands.

According to a further preferred embodiment, the filamentous receptor structure of the invention is attached to a carrier.

According to a further preferred embodiment, the filamentous receptor structure of the invention is stabilized against depolymerization.

According to a further aspect, the invention relates to a protein chip comprising several filamentous receptor structures of the invention, in which the filamentous receptor structures are attached to a carrier, preferably in a matrix arrangement.

### DEFINITIONS

An “isolated flagellar filament” is a flagellar filament which is present in a modified environment relative to its original (natural) environment, and which is not attached or bound to a membrane of a living cell. Such a flagellar filament might be a reconstituted flagellar filament or it might be produced by removing the cell-made flagellum, preferably recombinant flagellum, from the cellular membrane.

A “reconstituted flagellar filament” is a filament produced by the polymerization of identical or different flagellin proteins, as subunits, in vitro.

An “isolated flagellin” is a flagellin which can be found in an isolated flagellar filament, preferably in reconstituted flagellar filament, or which is in a monomeric form.

A “purified” protein, such as flagellin, flagellin fragment or flagellar filament, is a protein that was produced for subsequent application and in a form suitable for the application. In the process of the production, for example, the ratio of the purified protein and some other components of the sample containing the protein increased and it is suitable for the purpose of the application, it is preferably at least n %, where n is any integer between 20 and 100.

A “target substance” is a compound whose properties are studied, or which is to be detected in a sample or to be obtained from a sample, or one that is to be recognized or bound.

A target substance may be, for example, an atom, ion, simple or complex organic or inorganic molecule, including molecular ions, complexes or biological macromolecules or their structural units. A target compound might be bound to another substance, carrier, cell or cellular organ or organelle.

A “specific recognition” of a target substance by a protein is an event or series of events, where the target substance is contacted with the protein, and the strength of the interaction between them is different from that of the interaction which might formed or is formed between the same protein and at least one—preferably more than one—different target substance, and the protein under similar conditions. Preferably, the strength of the interaction between the protein and the target substance is higher, i.e. exceeds detection limit, than that of between the protein and a different target substance.

Highly preferably, in the course of specific recognition, among the studied target compounds there is a small number of target substances, preferably there is only one substance, which bind(s) to the protein capable of specific recognition more strongly than all the other target compounds studied.

A “reception” is a protein, protein fragment, protein subunit or supramolecular protein complex which is capable of the specific recognition and the binding of one or several target substances. Highly preferably, after the binding of the target compound the receptor provides means to detect the binding.

A “receptor protein” is a protein, which might be used as a receptor according to the definition above.

A “receptor subunit” is a receptor protein, which can function as a part of a supramolecular complex containing several proteins.

In the description, by the term “filamentous receptor structure” is meant an isolated, preferably reconstituted, flagellar filament that comprises at least one flagellin subunit, which is a receptor subunit.

By the “detection of binding” between the target substance and the receptor is meant the direct or indirect detection of the binding.

The “direct detection of binding” is a method, whereby, in the course of the binding of a target substance to the receptor capable its specific recognition, due to one or more binding event(s) a signal is produced which positively correlates with the number of binding events, and the occurrence of a binding event can be inferred by detecting the signal.

If quantitative value can be assigned to the said signal, said value can be either positive or negative.

In such a method, either the molecular environment altered due to the binding event can be detected, e.g. by any spectroscopical technique, or the bound target substance can be detected by immunological or microscopical techniques or the combination of them.

The definition does not encompass the methods of indirect detection of binding, thus in general, the methods and studies, wherein the generation of the sign is not due to the binding event(s), but it occurs or the signal arises far from the binding site or in a different site, i.e. not on the receptor protein or in its molecular environment and not on the bound target substance or in its molecular environment. In these cases the occurrence of the binding event could be deduced only in a speculative or indirect manner. Such excluded methods comprise e.g. competition studies, or when the concentration of the unbound target substance is measured in the solution.

By the “terminal regions” of flagellin are meant the N-terminal region and the C-terminal region which are conserved and show significant—preferably at least 20%, at least 30% or at least 40%—sequence identity in the flagellins of different species. The N-terminal region consists of typically about 160-200 or 170-190 or about 180 amino acids, the C-terminal region comprises about 80-120 or 90-110 or about 100 amino acids. The N-terminal regions can be also determined by aligning a known protein sequence, such as the flagellin sequence of Salmonella typhimurium [Smith and Selander (1990), sequence identification number of P06179 in the SwissProt database] with a flagellin sequence of an other species [e.g. Shigella flexneri, SwissProt Q08860], and those N-terminal and C-terminal regions having a sequence identity significantly larger than that of the other regions are considered as terminal regions.

The terminal regions or their significant portion play an important role in the polymerization of the flagellins into a flagellar filament.

The “variable region” of a flagellin is a region located between the terminal regions and preferably the region which—regarding the flagellins of different species—has a variability significantly higher than that in the terminal regions, i.e. the sequence identity among these sequences is typically very low. The variable region typically consists of the D3 domain and at least partly the D2 domain of flagellin.

The “D3 domain” of a flagellin is a domain of the variable region, which is located at the farthermost position from the flagellar filament axis and which has the largest exposed surface area (i.e. a surface in contact with the environment) among the flagellin domains. In Salmonella typhimurium the sequence of the D3 domain consists of approximately the 190-284 segment of flagellin. Typically, the major antigen determinant regions of the flagellar filaments can be found on the D3 domain.

The “hypervariable regions” of the flagellin D3 domain are the regions displaying high variability even between different strains of the same species, preferably, they show a variability higher than that of the other regions.

The “surface loop regions” of the flagellin D3 domain are loop regions that are in contact with the environment through the most extensive molecular surface, e.g. loop regions exposed to the solvent for the most part, among the loop regions. Highly preferably, by aligning these regions with the flagellin sequence of the Salmonella typhimurium, the regions matching the following regions are called surface loop regions: amino acids 205-213 (H1 loop), amino acids 236-244 (H2 loop) and amino acids 261-270 (H3 loop) of the S. typhimurium sequence. Furthermore, optionally these regions may comprise an additional 10, preferably 5, more preferably 3, 2, or 1 amino acid(s) in either direction.

The expression “the native fold is essentially retained” is meant as describing a situation, where the secondary structural elements and their arrangement relative to each other are essentially preserved.

Other terms used in the description are applied in the conventional manner or their meaning is obvious for a person skilled in the art.

### A BRIEF DESCRIPTION OF THE FIGURES

FIG. 1: Electronmicroscopic image of the Salmonella bacterium. Filaments of length of 5-20 μm are clearly shown.

FIG. 2: The subunit structure of flagellar filaments.

FIG. 3 (a): The structure of a flagellin subunit. (b) Arrangement of the flagellin subunits within the flagellar filament.

FIG. 4: A schematic diagram showing the structure of the D3 domain. The external surface of the domain is composed of three loops: loop 205-213 (H1, green), loop 236-244 (H2, blue) and loop 261-270 (H3, red).

FIG. 5: The subunit structure of a filamentous receptor composed of a single type of modified flagellin. The length of reconstituted filaments is well controllable. The distance between the binding sites on the surface of the filaments is approximately 5 nm.

FIG. 6: A filamentous structure prepared by co-polymerization of flagellin receptor subunits and Cys mutant flagellins. The Cys side chain on the external surface of the D3 domain facilitate simple immobilization of the filaments on a surface. Moreover, suitable reporter groups can be specifically attached to the Cys mutant subunits.

FIG. 7: Filamentous nanochip produced by block co-polymerization of several types of flagellin receptors and wild type flagellin. Each binding region is capable of recognizing and binding a different target molecule. The binding regions are separated from each other by regions of non binding (wild-type) flagellins. Isolation of nanochips can be facilitated by attaching a magnetic bead to the end of the filaments. The binding regions can be easily identified.

FIG. 8: A titration calorimetric study of the Ni-binding of a flagellin variant prepared by replacing the Leu209-Val235-Lys241-Ser264 side chains with histidine residues. The figure shows the titration calorimetry curve obtained by a MicroCal VPITC instrument and the respective calculated data.

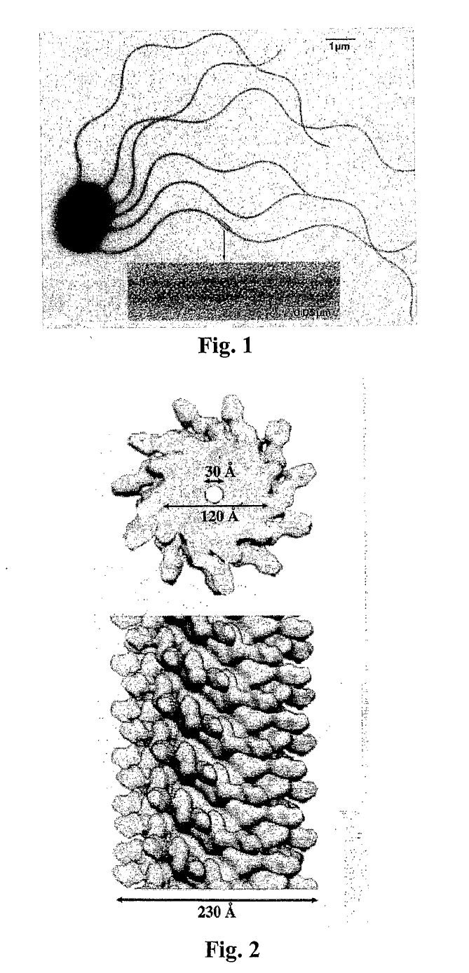

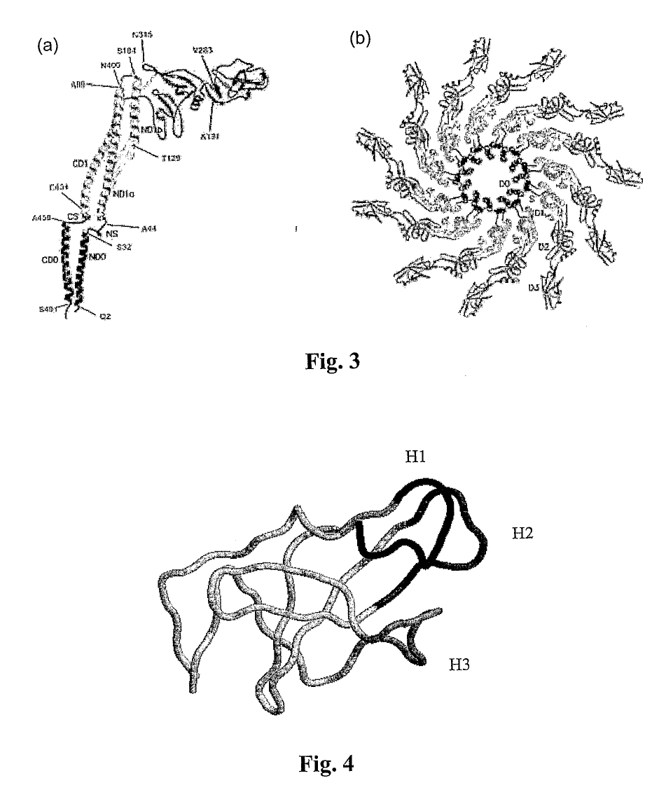

### A DETAILED DESCRIPTION OF THE EMBODIMENTS OF THE INVENTION

Certain embodiments of the invention are illustrated below in more details by way of examples. It will be apparent that on the basis of the teaching provided herein the person skilled in the art will be able to develop other embodiments of the invention without departing from the scope of the invention.

It will be understood that a flagellin from any species or strains might be used, provided that it meets the conditions required for a given application. These conditions will be apparent on the basis of the embodiments disclosed in the description. According to an embodiment, for example, the terminal regions of the flagellin of the invention must provide the polymerization ability. In the variable region, preferably in the D3 domain, there must be segments which can be altered without eliminating or decreasing this polymerization ability. According to another embodiment, within the variable region, preferably within the D3 domain, there are hypervariable regions which might be modified without disrupting the structure of other parts of the protein; for example, may be modified without disrupting the D3 domain as a whole. Examples for such flagellins are, for example, listed in the SwissProt database under the following identification numbers: P06179 (Salmonella typhimurium fliC-gene), P06175 (Salmonella rubislaw fliC-gene), P52616 (Salmonella typhiurium fljB-gene), PO₆₁₇₈ (Salmonella paratyphi-α, fliC-gene), P06176 (Salmonella cholerae-suis fliC-gene), P51615 (Salmonella abortusequi fljB-gene), P06177 (Salmonella muenchen fliC-gene), P04949 (Escherichia coli fliC-gene), Q06974 (Salmonella oranienberg fliC-gene), Q06973 (Salmonella Montevideo fliC-gene), Q06973 (Salmonella enteritidis fliC-gene), Q06968 (Salmonella berta fliC-gene), Q06971 (Salmonella dublin fliC-gene), q06983 (Salmonella senftenberg fliC-gene), Q06982 (Salmonella rostock fliC-gene), O52959 (Salmonella naestved fliC-gene), Q06981 (Salmonella moscow fliC-gene), Q06970 (Salmonella derby fliC-gene), Q06969 (Salmonella budapest fliC-gene).

If two or more of the above sequences are aligned, it will be apparent that even sequences with a relatively low percentage of overall identity (e.g. in 40-50%) may show high homology in their terminal regions. Thereby, terminal regions and variable regions can be distinguished. On the basis of sequence homology (sequence identity or -similarity), further flagellins, useful in the present invention, can be identified. Among these further flagellins there might be ones which are encoded not by the fliC gene (i.e. 1^{st} phase flagellins) but, for example, by the fljB gene (i.e. 2^{nd} phase flagellins) or by other genes (e.g. fliA). As a matter of course, the criteria according to the invention must be met.

When further flagellins are to be identified, it is particularly useful to search for sequences homologous to the terminal regions. Either amino acid sequences or nucleotide sequences might be used for finding further homologous sequences. A number of flagellin sequences and sequences of genes encoding flagellins are available, for example, at the EMBL (European Molecular Biology Laboratory, European Bioinformatics Institute, http://www.ebi.ac.uk/), for example, in the following databases: SwissProt, trEMBL, ill. EMBL Nucleotide Sequence Database. The sequence of the Salmonella typhimurium fliC gene (1^{st phase} flagellin) is available, for example, under identification numbers D13689 and AY649719, while the sequence of the fljB gene (2^{nd} phase flagellin) can be found e.g. under the identification number of AF045151.

A search for related sequences and the pairwise sequence alignments may be performed by using, among others, different versions of the BLAST algorithm (see e.g. http://www.ebi.ac.uk/blast/index.html) or of the FASTA algorithm (see e.g. http://www.ebi.ac.uk/fasta/index.html). For aligning and comparing several sequences different versions of the CLUSTAL program are considered to be appropriate (see e.g. http://www.ebi.ac.uk/clustalw/index.html).

Thus, by using well-known sequences, the person skilled in the art will be able to identify, without difficulty, flagellin genes of other species expressing the flagellin protein, e.g. species moving with flagellum (e.g. Flagellata). Besides the species mentioned above, such species might be, for example Escherichia species, Shigella species, Serratia species, Pseudomonas species, Vibrio species, Campylobacter species, Helicobacter species, Bordatella species, Leptospira species, Wolinella species, and e.g. the Aquifex species from among the thermophylic bacteria, etc.

By comparing flagellin encoding genes between strains of a given species, the hypervariable regions within the variable regions, i.e. regions showing higher variability than that of the variable regions, can be preferably determined [see He, X. et al. (1994); Frankel, G. et al. (1989)]. These regions are especially suitable for constructing binding sites, as described below.

Preparation of Flagellin-Based Filamentous Receptors

The aim of the present inventors was to prepare flagellin-based artificial receptors by modifying the variable regions, preferably the D3 domain, in a way preserving the polymerization ability of flagellin.

According to an embodiment, the complete D3 domain is replaced with a protein domain comprising a desired binding site. This protein domain may be any domain carrying a binding site. By replacing the D3 domain in the flagellin sequence with the sequence encoding this protein domain in question, and by expressing the construct as a fusion protein, a domain with native structure (tertiary structure) can be obtained. The size of this domain will not disturb the polymerization of the flagellin. Such a domain might be, e.g. a binding domain prepared from the above mentioned fibronectin domain or the so-called “anticalins” produced from a lipocalin protein [Xu, L. et al. (2002); Skerra, A. (2001)], and other binding domains, preferably of natural origin.

The polymerization ability of flagellin is usually preserved even if the D2 domain is involved in the replacement (though the stability of the filaments might be decreased). Therefore, any binding-domain replacement in the variable region is considered to be within the scope of the invention.

According to a further embodiment artificial receptors can be prepared in various ways, using the D3 domain as a scaffold, for example as follows:
the amino acid sequence of the H1, H2, H3 loop regions might be altered by using artificial evolution methods
the binding properties of the H1, H2, H3 loop regions may be modified by directed mutagenesis, by protein engineering, and by inserting binding segments found in other proteins or by inserting side-chains having the desired property.
by attaching proteins and other molecules with specific binding characteristics to the surface of D3 domain.

The above mentioned three surface regions of the Salmonella typhimurium flagellin significantly overlap the hypervariable region. Provided that the structure of a given flagellin is known, (and probably a number of structures will be solved in the future), the person skilled in the art will be able to identify the loop regions and by modifying them can develop a binding site according to the invention.

Provided that the three dimensional conformation is unknown, the necessary alterations should be performed in the variable regions which can be identified as explained above [He, X. et al. (1994); Frankel, G. et al. (1989)].

Receptors Based on the D3 Domain

The present inventors found that the D3 domain has a stable structure that might serve as scaffold for artificial receptors. The D3-based receptors promise several advantages over the IgG molecules, as they are stable, small in size, and can be produced in bacteria easily and inexpensively.

A comparison of flagellins from different bacteria also suggests that D3 domains are excellent scaffold structures [Smith, N. and Selander, R. K. (1990); Joys, T. M. (1985); Wei, L. N. and Joys, T M. (1985)]. When comparing the amino acid sequences in the D3 domains of flagellins of different origin large insertions/deletions can be observed in the surface loop regions, resulting in the high variability of the size of the D3 domain. Since the flagellar filaments are located outside the cell and they are easily accessible, they function as the major antigen determinants of the bacteria [Macnab, R. M. (1995)]. Having the ability to alter the surface characteristics of the D3 domain forming the outermost part of the filaments means a selection advantage for the bacterium, whereby, it is capable of making itself hardly recognizable and, as a consequence, making the immune response less expressed.

It is feasible to perform the directed evolution studies with a suitable flagellin fragment (such as the D2-D3 or the D3 domains), then the gene segment encoding the proper variant(s) capable of recognizing and binding the target substance should be re-inserted into the complete flagellin gene or into a larger fragment thereof that is appropriate for receptor production.

Directed Evolution Methods

The amino acid sequences of the variable regions and preferably of the H1, H2, H3 loop regions can be modified by artificial evolution methods [Brakmann S. et al. (2002), Amstutz, P. et al. (2001), Takahashi, T. T. et al. (2003); Fernandez-Gacio, A. et al. (2003); Tao, H. and Cornish, V. W. (2002), Ladner, R. C. et al. U.S. Pat. No. 5,837,500, Jermutus et al. (2001), Mössner, E., Pluckthun, A, (2001), and Pluckthun et al., U.S. Pat. No. 6,589,741]

The mutations in the directed evolution can be performed by using, for example, any of the following methods: “error-prone” mutagenesis [Cadwell et al. (1994)], “DNA-shuffling” mutagenesis [Stemmer, (1994)], or PCR-cassette mutagenesis [Virnekäs et al. (1994)].

Several examples are known where artificial receptors capable of recognizing and binding certain target molecules were produced by using directed evolution methods in which the amino acid sequences of surface loop regions of proteins originally not having a receptor function were varied [Xu, L. et al. (2002); Skerra, A. (2001); Nygren P. and Uhlen, M. (1997); McConnell S., Hoess, R. H. (1995); Ku, J., Schultz, P. G. (1995)]. By applying a similar strategy to the D3 domain of the flagellin, numerous mutants can be prepared by varying the amino acid sequences of the H1, H2 and H3 surface loop regions resulting in different in vivo or in vitro libraries [see e.g. Arnold F. H. and Georgiou G. (Eds.) (2003)]. The applied library might be bacterial or yeast library, phage display library, yeast two-hybrid system, ribosome display library [see e.g. Plückthun et al., U.S. Pat. No. 6,589,741, Mattheakis et al., (1994); Hanes et al., (1997); Amstutz et al., (2001)], mRNA fusion library [see e.g. Roberts et al., (1997) or e.g. Amstutz et al., (2001)], or a protein fragment complementary library [see e.g. Mössner and Plückthun, (2001)].

The production of mutant genes may be performed by the method described by Skerra in the case of the lipocalin protein [Skerra, A. (2001)]. Mutants capable of binding the desired target molecule might be preferably selected from the created numerous variants by using the ribosome display [Amstutz, P. et al. (2001)] or the mRNA display methods [Takahashi, T. T. et al., (2003)]. Since these methods contain exclusively in vitro steps, a mutation cycle can be carried out in a relatively short period of time and with a library having a relatively large number of members.

Construction of a Site Capable of Binding Conjugates or Allowing Immobilization to a Carrier Surface

In a simple case, a side chain, suitable for conjugating a molecule carrying a specific recognition site, is introduced on the surface of the D3 domain by a single amino acid replacement.

The wild type flagellin protein, for example, does not contain cysteins. By the use of site-directed mutagenesis, one or more amino acids in the H1, H2 or H3 loop regions might be replaced with a cysteine, which results in modified flagellins that contain a D3 domain carrying easily accessible surface SH-groups. The inventors successfully replaced e.g. Thr239 of the H2 loop with a cysteine residue. The obtained Cys239 mutant flagellin can be easily labelled with different compounds, such as fluorescent stains, specific to thiols.

D3 domains are located on the external surface of the filaments. Therefore, filaments constructed from modified flagellin subunits containing a cysteine side-chain in their D3 H1, H2 or H3 loop regions, or filaments containing such subunits can be simply attached to suitable carrier surfaces through the reactive thiol group, using well-known methods. As a matter of course, other well known techniques may also be used to construct a site suitable for covalent binding.

Another example is the construction of a metal binding site created by simple amino acid replacements in the D3 domain. In the knowledge of the size of the metal ion, using computer assisted graphics and molecular modeling [e.g. by any of the Protein Explorer, DeepView (or SwissPDB Viewer) or the WPDB programs], amino acid residues present in a suitable orientation and distance are identified. Afterwards, by site specific mutagenesis, these residues are replaced with ones that are capable of complexing the given metal ion. Thus, a site capable of binding metal ions can be constructed.

In some natural proteins there are certain metal ion binding sites, such as the Ca²⁺, Mg²⁺, Fe²⁺, Zn²⁺, Ni²⁺ binding sites, which might be considered as models. Moreover, more or less aspecific binding sites may be constructed, which are capable of binding several kinds of ions, e.g. several kinds of heavy metal ions.

Such chelating residues are, for example, residues containing a carboxyl group (aspartic acid, glutamic acid), an electron donor group (e.g. serine, threonine, cysteine, asparagine, glutamine) or histidine which comprises an imidazole group.

If desired, a suitable binding site can be provided by attaching a binding domain or a prosthetic group to the surface of the D3 domain.

Filamentous Receptor Structures

Compared with the known artificial protein receptors particular advantages are expected from the production and use of flagellin receptors modified in their D3 domain as described above, because they can be used to construct various filamentous receptor structures.

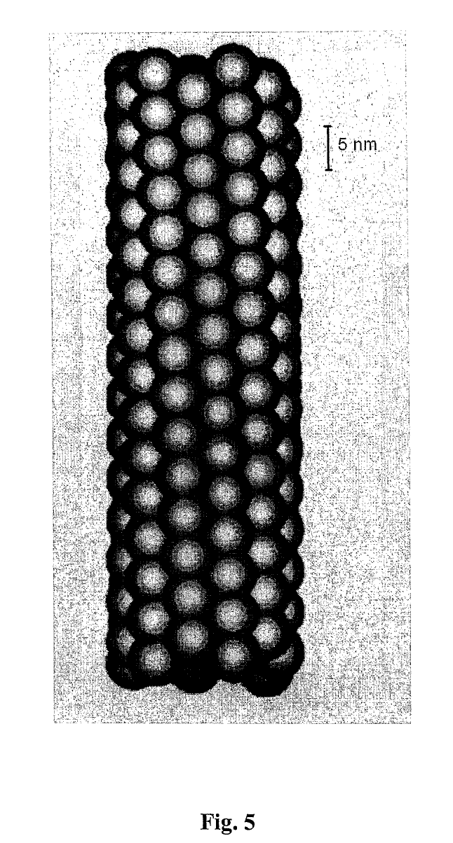

(a) Constructs Based on a Single Type of Flagellin Receptor (FIG. 5)

By precise control of the polymerization process, filament structures with a desired length (0.1 μm-10 μm in length) can be constructed from a single type of flagellin, which might contain several hundreds or even several tens of thousands of subunits. On the surface of the filamentous receptors binding sites of high density can be obtained, whereas the distance between the individual binding sites is about 5 nm. Thus, no special carrying matrix is necessary for the spatial localization of the subunits with a suitable density. A further advantage is that the receptor subunits forming the filamentous structure have identical local environments. Even in the case of ligands with small molecular mass (such as the heavy metal ions) the high binding site density seems to be promising for detecting the binding event and renders the binding of compounds present in a low concentration possible.

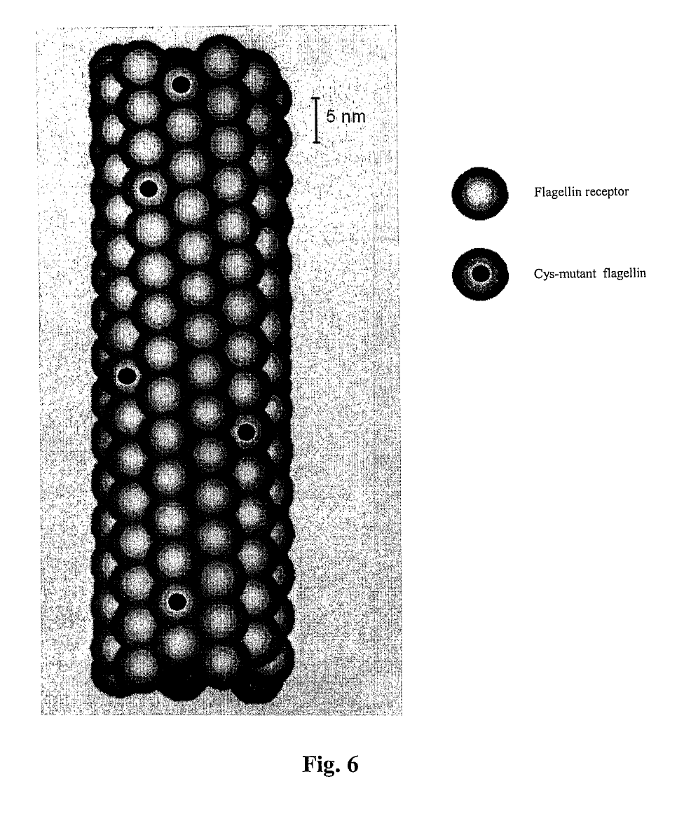

(b) Mixed Polymerization of Several Types Off Flagellins (FIG. 6)

It is reasonable to perform a mixed polymerization, in an appropriate ratio, of flagellin receptors and modified flagellins which help, for example, to attach the filamentous structures to the surface of a sensor chip or to detect the binding process. By using site-directed mutagenesis a flagellin variant can be prepared which comprises a single inserted cysteine side chain (reactive SH-group) on the external surface of its D3 domain. If a mixed polymerization is performed with the Cys mutant flagellin and other flagellin receptors (for example in a ratio of 1:100), the resulting filamentous receptors will have randomly distributed SH-groups on their surface. (The frequency of occurrence of SH-groups can be controlled by changing the ratio.) Immobilization of the filamentous receptor structures to the surface of a sensor chip can be easily achieved through these surface-exposed SH-groups. Moreover, by attaching a suitable reporter molecule (such as a fluorescent dye) to the SH-group, a detecting subunit surrounded by receptor subunits can be created. The spectral properties of the detecting subunits will change due to the binding events occurring in their environment, which may even allow the monitoring of the binding process in real-time.

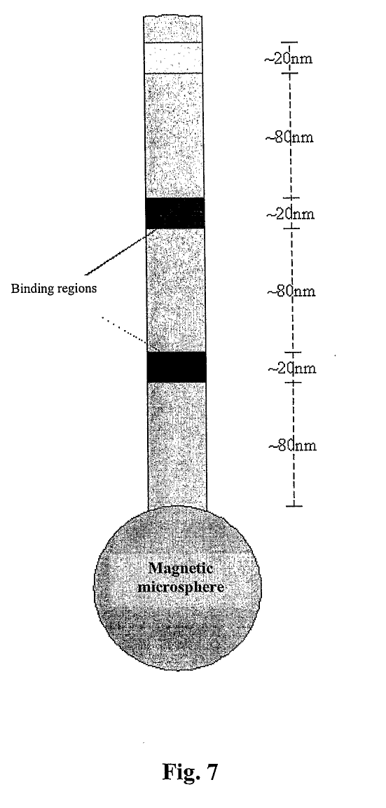

(c) Block Co-Polymerization of Several Types Off Flagellins (FIG. 7)

Filamentous receptors (nanochips) containing regions with different binding properties can also be prepared by controlled block co-polymerization of at least two types of flagellins of different binding properties. Optionally, these binding regions might be separated from each other with regions of wild-type non-binding flagellins. The length of these regions can be precisely controlled. These structures bind the individual target molecules of a multicomponent mixture at different positions. By using a suitable detection method, the composition of the mixture might be deduced from the binding positions.

An exemplary embodiment of the filamentous receptor structures prepared by block co-polymerization is the following:

Short segments of filaments are prepared from wild-type (unlabelled) flagellin, and they are covalently attached to the surface of magnetic microbeads. A thin layer (˜20 nm) of FR₁ flagellin receptors capable of binding the TH1 target molecule followed by a thicker (˜80 nm) layer of wild type flagellin is polymerized to the end of the filaments. The procedure is repeated with several types of flagellins (FR₂, FR₃, . . . FRₙ; n˜20-50) capable of recognizing different target molecules. Thus, finally a filament is obtained (nanochip), which comprises well-separated binding regions with different specificity. A flagellum comprising 20 to 50 different binding regions can function as a parallel ELISA-like method. The nanochips will bind the target molecules in the sample studied; then they can be isolated easily with a magnet. Detection may also be easily performed by electronmicroscopy, if the target molecules to be studied are attached to the surface of nanogold particles, or if the target molecules bound to the filaments are labelled with secondary flagellin receptors conjugated to nanogold particles.

As a matter of course, the number and arrangement of the binding regions can be varied just as their length (their “thickness”) or the distance between them.

For example, depending on the length of the filament and on the length and the density of the binding regions even several hundred, preferably, at most 200 binding regions can be constructed on a long filament. Highly preferably, the number of the binding regions is 5-150, 10-100, 15-75, 20-50, etc.

The length of the binding regions might be preferably 10-100 nm, 15-50 nm, 20-30, e.g. about 15, 20, 25, nm.

The person skilled in the art will understand that the optimal length (thickness) of the binding regions and the distance between them may depend on the target molecule to be recognized, the type of the receptor subunit and the binding site, the mode of the detection, etc. Being aware of the particular task, a skilled person can easily determine these parameters.

Regions composed of detecting subunits might also be located right beside the binding regions or in a given distance from them, as outlined above.

The embodiments (a)-(c) disclosed above can be combined with each other. Provided our filamentous receptor structures are attached to an appropriate surface and by selecting a suitable detecting method they can function as basic elements of biosensors.

Immobilization of Filamentous Receptors

A particularly preferred method for binding as well as constructing filamentous receptors on the surface of a sensor chip is the following. As a first step, short pieces of filaments are covalently attached (e.g. through cysteine mutant subunits) to the surface of the sensor chip; then, one after the other, solutions containing the respective modified flagellin subunits are circulated above the sensor chip. Thereby, the desired filamentous structures can be constructed in situ according to any of the (a)-(c) methods. In the art there are several well known methods for immobilizing biomolecules [see e.g. Cass, T. (1998); Aslam M. and Dent A. (1998)].

Stabilization of Filamentous Structures

At ambient temperature the flagellar filaments are stable for a long period of time and they are resistant to proteases. The only event that might endanger their integrity is the slow depolymerization of subunits at the distal end of the filaments. To prevent this event it is feasible to produce mutant flagellin subunits that contain cysteine side chains in relevant positions to spontaneously form disulphide bridges along the protofilaments or between the subunits located in the adjacent protofilaments. If filamentous receptors are constructed from such type of flagellins or if such flagellin subunits are polymerized in a thin layer to the end of filamentous structures, depolymerization of the filaments can be blocked. These receptors can be applied in a wide range of conditions.

Based on the atomic structure of the Salmonella typhimurium flagellar filament (Yonekura et al., 2003) amino acid pairs of appropriate distance can be found in the adjacent subunits by computer-aided molecular modeling. The appropriate distance between the Cα atoms should fall into the range of 5-7 Å and the side chains of the chosen amino acids should face towards each other. Replacing these side chains with cysteines, disulphide bridges may be formed between adjacent subunits, e.g. along the protofilaments disulphide bridges can be created by the replacement of Gly133 and Asn315 amino acids with cysteines, and between the protofilaments disulphide bridges can be formed by the replacement of Ile319 and Ala413 amino acids with cysteines. Because positions in the sequence, which seem to be promising for the formation of disulphide bridges between subunits, might be partly found in the conserved regions, the amino acid replacements might cause the loss of the polymerization ability. A study of several promising double cysteine mutants should provide empirical evidence allowing the skilled person to decide whether the modified forms to be constructed preserve their ability to polymerize and whether the stabilizing crosslinks could really be established or not. The end of native filaments is covered by a molecular cap with a pentamer structure comprising a few copies of the HAP2 protein (Yonekura et al. (2000)) effectively blocking the depolymerization of the flagellin subunits (Diószeghy et al. (2004)). Therefore, the reconstituted filaments can also be stabilized by adding HAP2 protein to the filaments, thus, blocking the depolymerization by way of binding to the end of the filaments.

The two methods might even be combined. Thus, for example the flagellar filament can be stabilized by forming disulphide bridges between the HAP2 proteins and the flagellin subunits by introducing cysteine side chains in appropriate positions.

Detection of Binding of the Target Molecule

The detection of the binding of the target molecules can be performed by the above mentioned electronmicroscopical method. There is also a possibility for reporter molecules, e.g. chromophores, such as fluorescent molecules, to be inserted in the vicinity of the binding sites, and due to the binding event their spectroscopic characteristics will change, whereby the binding can be detected. The reporter molecules might be placed in the same flagellin subunit or in an other flagellin adjacent to the binding site. The optimal arrangement depends on the target substance, e.g. its size, spectroscopical properties, etc.

According to another embodiment, the receptors are attached to the surface of a sensor chip (or cantilever oscillator), then the change in the surface properties (such as the refractive index) or in the mass of the reed caused by the binding of the target molecule is measured.

Detection might be performed, for example, by:

surface plasmon resonance spectroscopy [Rich, R. L. and Myszka, D. G. (2000). Myszka, D. G. and Rich, R. L. (2000)],

cantilever oscillator sensors [Ilic, B., et al. (2004)],

optical wave-guide sensors [Wybourne, M. N. (1995) U.S. Pat. No. 5,465,151]

The techniques and methods disclosed below are only examples of illustrating the embodiments of the invention.

### EXAMPLES

### 1. Preparation of Flagellin Receptors by Modifying the D3 Domain

### (a) Preparation of Artificial Flagellin-Based Receptors by Directed Evolution

It was found that the D3 domain preserves its integrity and structural stability even in its isolated form. The gene segment encoding the D3 domain was inserted into an expression vector, then E. coli K12 cells were transformed with this construction and the polypeptide chain of the D3 domain was produced in large quantity. The results of the CD-spectroscopy and the scanning microcalorimetry performed on an isolated D3 fragment clearly demonstrated that the isolated D3 domain had a stability and secondary structure typical of the native structure. Therefore, the directed evolution studies can be successfully run on the D3 domain consisting of only 94 amino acids. In the present example a ribosome display method is used. Preferably as the example the method developed by Plücktun et al. is applied with the necessary modifications, which are obvious for a skilled person [Jermutus et al. (2001) and Plückthun et al., U.S. Pat. No. 6,589,741].

A DNA sequence encoding the D3 domain of the Salmonella typhimurium or a segment shorter or longer with a few amino acids is constructed by PCR. Then DNA fragment variations encoding modified D3 are produced from nucleotide sequence segments encoding H1, H2 and H3 loop regions by random mutagenesis, e.g. error-prone” PCR method [Zaccolo, M., et al. (1996)]. From the so obtained fragments a DNA library is prepared. The random mutagenesis might be carried out individually on each of the segments encoding H1, H2 or H3, or it can be performed on several segments at the same time.

Messenger RNA is produced from the DNA fragments and ribosome display library is constructed by the method of Jermutus et al. [Jermutus et al. (2001) and Plückthun et al., U.S. Pat. No. 6,589,741]. After that a segment encoding a C-terminal spacer polypeptide is fusioned in phase to the mRNA segments encoding the D3 by PCR. During the transcription the spacer occupies the ribosome tunnel and anchors the protein produced, thereby making the formation of protein 3D structure possible.

Ribosome-bound complexes comprising as a fusion the mRNA and the D3 domains (polysome) are selected with respect to the affinity to a given target molecule. In the present example the target molecule is a relatively small molecule, e.g. hapten, e.g. fluorescein. The selection might be performed, for example, in a way that the target molecule is conjugated to a protein, e.g. BSA (bovine serum albumin), and complexes bound to this conjugate are selected [Beste et al., (1999)]. Another possibility is, for example, the biotinylation of the target molecule; the complexes bound to the given construct to a satisfactory extent are then isolated by using streptavidine coated magnetic beads [see e.g. the method of Jermutus et al. (2001)]. DNA is amplified by reverse transcription PCR from the mRNA encoding the selected proteins, then the amplified DNA is mutagenized again, finally the obtained DNA fragments are subjected repeatedly to the selection cycle.

According to a preferred variation of the method, the steps of mutagenesis and the selection might be repeated in a way that the mutagenesis is performed not only in any of the H1, H2 and H3 loop regions but also in the complete D3 domain. Reasonably, this further mutagenesis is done after a certain level of affinity has been reached. As a result, the affinity of the mutants should possibly increase.

When an appropriate D3 domain or the library containing it is obtained, it is cloned into a suitable construct.

D3 domain variant(s) capable of recognizing and binding the target molecule obtained at the end of the selection, might be used as receptor(s), or by inserting the gene segments encoding the variant(s) into the complete flagellin gene, consequently, flagellin variant(s) having appropriate binding properties and ability to polymerize can be produced. Then, flagellar filaments produced from the flagellins obtained this way might be used as receptors.

The library of the present example can be stored at any step of the method and might be used later for the selection of other compound(s).

### (b) Construction of Flagellin Variants by Inserting Side Chains or Segments Having Desirable Binding Properties

The Thr293 in the H2 loop region of the D3 domain of the Salmonella typhimurium flagellin protein was replaced by cysteine, using site-directed mutagenesis. Our results show that the SH group is easily accessible and desired molecules can be bound there. Methods for the production of this type of mutants and similar mutants are well known in the [see e.g. Sambrook D. and Russel D. W., Molecular Cloning. A Laboratory Manual, (2001)].

Different molecules carrying binding sites, e.g. proteins, might be conjugated to the inserted SH group. Such proteins are, for example, Fab-, Fᵥ-fragments, V_{H}- or V_{L}-domain of immunglobulin molecules, certain binding proteins, e.g. receptor proteins, nucleic acid binding proteins, domains that bind Ca²⁺ or other metal ions, etc., or e.g. small molecule binding proteins that were modified in favour of binding the given target molecule [see e.g. Weiss, G. A. and Lowman, H. B. (2000); Nygren, P., and Uhlén, M., (1997)].

Segments observed in other different proteins can be inserted into the H1-H3 loop regions of the D3 domain by using conventional genetic engineering techniques (see e.g. methods disclosed in the following publications: Wu, J., Y., et al. (1989), Stocker B. A. and Newton S. M. (1994), Marjarian et al., EP 0419 513]

### c) Construction of Flagellin Variants that Bind Ni Ions

In Ni binding proteins occurring in nature, the coordination of the Ni ions is usually performed by the imidazole groups of several (2-4) histidine side chains. By computerized graphics and molecular modeling, the inventors checked the variable D3 region of the flagellin to identify amino acids having side chains in suitable orientation and distance for constructing metal ion binding centre by replacing the relevant amino acids with histidine. The following side chain combination were decided to be replaced by histidine:

a) Leu 209, Val 235, Lys 241

b) Leu 209, Val 235, Lys 241 and Ser 264

c) Leu209, Gly211 and Lys 241

d) insertion of a (His)₆ hexapeptide between the Thr239 and Gly240 amino acids located on the external surface of the D3 domain.

The proposed His-binding variants were constructed by directed mutagenesis. The mutant genes were sequenced and transformed into a flagellin-deficient SJW2536 Salmonella strain. In every case the mutant flagellins were produced in large quantities and efficiently exported from the bacterial cells. The mutant flagellins retained their ability to polymerize, and by the addition of ammonium sulphate they produced filaments with a morphology identical with that of the native filaments.

At the first approach, the Ni binding ability of the produced flagellin variants were checked in Ni-chelate affinity chromatography by using Pharmacia HiTrap Ni-sepharose column that selectively binds the proteins having suitably oriented clusters of His side chains on their surface. The bound proteins were eluated with imidazole and analyzed in SDS-PAGE. It was found that while the (His)₆ variant (d) was bound to the column strongly, the others did not bind significantly. These results meet our expectations, since the (His)₆ variant was planned to construct a surface Ni binding site, while the other mutants are probably embedded inside the D3 domain providing a binding site accessible only by the free Ni ions (through diffusion).

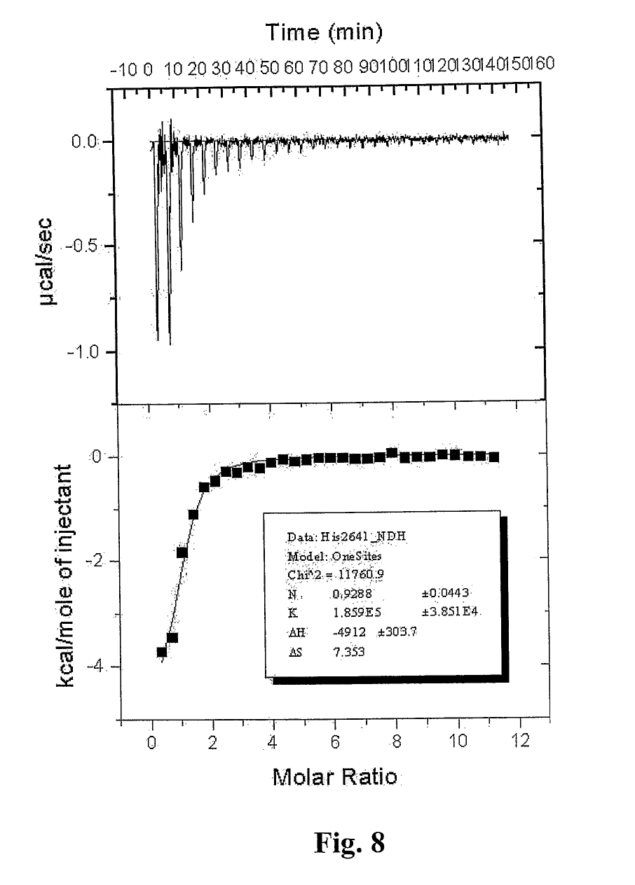

The quantitative analysis of the Ni binding ability of mutants was performed by isothermal titrating calorimetry in a MicroCal VPITC device. A variant constructed by replacing the Leu209-Val235-Lys241-Ser264 side chains with cysteine (variant b) showed the strongest Ni binding (FIG. 8) with a dissociation constant of K_{d}=5 mM, and one Ni ion was bound to one flagellin subunit. In the case of the (His)₆ variant, the binding was weaker by an order of magnitude (K_{d}˜50 mM), however, 2 Ni ions were bound to a subunit on average.

The desired filamentous structure can be constructed from the modified flagellin produced in this example as taught herein.

### 2. Expression and Purification of Flagellin Receptors

The constructed genes encoding flagellin receptors were ligated to pBR322-vector. The recombinant plasmids were transformed to flagellin deficient SJW2536 Salmonella (or E. coli) cells by electroporation. The flagellins produced appeared in large quantities on the surface of the cells as filaments and could be easily broken off with mild mechanical action. Then the filaments could be isolated in a few simple steps without lysing the cells [Wakabayashi, K. (1969)]. It was found that about 50-60 mg flagellin could be produced from a 2-liter culture. Optionally, the produced flagellin proteins could be further purified by ion exchange chromatography [Vonderviszt, F. at al. (1989)]. The pure flagellin preparates can be stored at −20° C. for years.

### 3. Construction of Filamentous Structures

Filamentous structures might be constructed from flagellin receptors. The production of filaments can be initiated by the addition of short filament segments [Asakura, S. (1970)] or agents inducting precipitation (such as the ammonium sulphate) [Asakura, S. et al. (1964)]. The polymerization of the flagellin can be easily controlled.

(a) Polymerization of the Flagellin by the Addition of Ammonium Sulphate

By adding 3.5 M ammonium sulphate (AS, in a final concentration of 1.0 M) to a flagellin solution, (5 mg/ml) prepared in Tris-HCl buffer (20 mM, +150 mM NaCl (pH=7.8); herein below: Tris/NaCl buffer) the monomers polymerized in 1 hour, and the average length of the produced filaments was 300 nm [Yamashita, I. (1991)]. By decreasing the amount of AS to be added, longer and longer filaments could be produced, thus, for example, at the final AS concentration of 0.5 M the average length of the produced filaments was 2 μm [Yamashita, I. (1991)]. The obtained filaments could be easily separated from ammonium sulphate by high speed centrifugation (˜100000 g) and the filament precipitate was resuspended in an appropriate buffer.

By polymerizing a solution, containing several types of flagellin receptor subunits in a desired ratio, with ammonium sulphate, such filaments might be obtained that have a structure containing randomly distributed flagellin variants in a localization ratio corresponding to the initial ratio numbers.

(b) Polymerization to the Ends of Short Filament Pieces

The polymerization of the flagellin monomers always takes place only at one end of the filaments [Asakura, S. et al. (1968)]. When flagellin monomers are added to short filament pieces in Tris/NaCl buffer, the monomers will be spontaneously incorporated—even without the addition of a precipitating agent—in the very end of the filament which is capable of growing. By controlling the amount of flagellin added and the polymerization time, the growing rate of the filaments and the length of the developing filaments can precisely be controlled. Short filaments functioning as polymerization cores, might be produced by the ultrasonic treatment of flagellin pieces broken off the bacterium or by the polymerization of flagellin monomers in the presence of precipitation agent in high concentration (e.g. 1.5 M AS) [Asakura, S. (1970); Asakura, S. et al. (1964)].

For the block co-polymerization of the flagellin receptors, at first a solution containing short filaments is prepared by adding AS solution (4 M, in a final concentration of 1.5 M) to the monomer solution (3 mg/ml). The produced filaments are centrifuged (300000 g, 15 min), then the pellet is resuspended in Tris/NaCl buffer. By adding type 1 flagellin receptor monomers (3 mg/ml) to the filaments and by controlling the polymerization time a layer of desired thickness can be built onto the end of the filaments. The filaments are separated from the monomer subunits by centrifugation, and the pellet is resuspended. Then, by adding type 2 flagellin monomers to the filaments in the way described above, a type 2 layer of desired thickness can be built onto the end of the filaments. By repeating the method several times, a filamentous structure might be obtained, along which segments with different properties, e.g. binding properties alternate.

### INDUSTRIAL APPLICABILITY, THE ADVANTAGES OF THE INVENTION

The flagellin molecule is a particularly promising object for creating artificial receptors. It has several advantageous properties as compared to the scaffolds used so far:

Flagellin receptors can be produced in bacteria; they appear as filaments on the surface of the cells, thus, they can be purified in large quantities without lysing the cells.

From flagellin receptors filamentous structures can be constructed. Along the filament, regions providing different functions can be constructed from flagellin monomers of different properties.

Extremely high receptor density can be obtained on the surface of the filament.

The filamentous form is resistant to proteases. Moreover, for example, in the case of Salmonella typhimurium flagellin it is stable under 40° C.; the thermal stability can be significantly increased provided that the flagellins are obtained from thermophylic bacteria.

Filaments of desired length can be produced by changing the conditions of polymerization.

### REFERENCES

Amstutz, P. at al. (2001) In vitro display technologies: novel developments and applications. Curr. Op. Biotech. 12, 400-405.
Arnold F. H. and Georgiou G. (Eds.) (2003) Directed evolution library creation. Methods and Protocols. Methods in molecular biology, Vol 231; Humana Press
Asakura, S. (1970) Polymerization of flagellin and polymorphism of flagella. Adv. Biophys. 1, 99-155.
Asakura, S., Eguchi, G. and Iino, T. (1964) The reconstitution of bacterial flagella in vitro. J. Mol. Biol. 10, 42-56.
Asakura, S., Eguchi, G., Iino, T. (1968) Unidirectional growth of Salmonella flagella in vitro. J. Mol. Biol. 35, 227-236.
Aslam M. and Dent A. (1998) Bioconjugation. Protein coupling techniques for the biomedical sciences., Chapter 8; Macmillan Reference Ltd.
Beste G., Schmidt F. S., Stibora T., Skerra A., (1999) Small antibody-like proteins with prescribed ligand specificities derived from the lipocalin fold. Proc Natl. Acad. Sci. USA, 96, 1898-1903.
Brakmann S., Johnson K. and Johnsson K. (2002) Directed molecular evolution of proteins. John Wiley and Sons
Cadwell R. C., Joyce G. F. (1994) Mutagenic PCR Method Appl, 3(6), 136-140
Cass, T. (1998) Immobilized biomolecules in analysis. A practical approach. Oxford University Press Diószeghy Z., Závodszky P., Namba K., Vonderviszt F. (2004) Stabilization of flagellar filaments by HAP2 capping. FEBS Letters 568, 105-109.
Fernandez-Gacio, A., Uguen, M. and Fastrez, J. (2003) Phage display as a tool for the directed evolution of enzymes. Trends Biotechnol. 21, 408-414.
Frankel, G., Newton, S. M., Schoolnik, G. K., Stocker, B. A., (1989) Mol. Microbiol, 3(10) 1379-1383
Hanes J., Pluckthun A., (1997) In vitro selection and evolution of functional proteins by using ribosome display. Proc Natl Acad Sci USA. 94(10) 4937-4942
He, X., Rivkina, M., Stocker, B. A. D., and Robinson, W. S., (1994) J. Bacteriol., 176(8), 2406-2414;
Ilic, B., Craighead, H. G., Krylov, S., Senaratne, W., Ober, C., Neuzil, P. (2004) Attogram detection using nanoelectromechanical oscillators. J. Appl. Phys. 95, 3694-3703.
Jermutus, L., Honegger, A., Schwesinger, F., Hanes, J., Plückthun, A. (2001) Tailoring in vitro evolution for protein affinity or stability, Proc. Natl. Acad. Sci. USA, 98(1), 75-80
Joys, T. M. (1985) The covalent structure of the phase-I flagellar filament protein of Salmonella typhimurium and its comparison with other flagellins. J. Biol. Chem. 260, 15758-15761.
Ku, J., Schultz, P. G. (1995) Alternate protein frameworks for molecular recognition. Proc. Natl. Acad. Sci. USA 92, 6552-6556.
Ladner, R. C., Gutterman, S. K., Lindsay, B., Markland R. W., Ley A. C., Kent R. B., (1998) U.S. Pat. No. 5,837,500
Macnab, R. M. (1995) Flagella and motility. In E. coli and Salmonella. Cellular and molecular biology. (ed.
Neidhart, F. C.) pp. 123-145, American Society for Microbiology.
Marjarian, W. R., Stocker, B. A. D, Newton, S. M. C., (1989) Recombinant flagellin vaccines EP 0 419 513
Mattheakis L. C., Bhatt R. R., Dower W. J., (1994) An in vitro polysome display system for identifying ligands from very large peptide libraries. Proc. Natl. Acad. Sci. USA 91, 9022-9026
McConnell S., Hoess, R. H. (1995) Tendamistat as a scaffold for conformationally constrained phage peptide libraries. J. Mol. Biol. 250, 460-470.
McCoy J. M. and Lu, Z., (1995) Method of detecting ligand interactions. International publication No. WO 9534664
Metzger, H. (1990) Fc receptors and the action of antibodies. American Society of Microbiology.
Mimori-Kiyosue, Y., Vonderviszt, F. and Namba, K. (1997) Locations of terminal segments of flagellin in the filament structure and their roles in polymerization and polymorphism. J. Mol. Biol. 270, 222-237.
Mössner, E., Plückthun, A, (2001) Directed evolution with fast and efficient selection technologies Chimia 55, 324-328
Myszka, D. G. and Rich, R. L. (2000) Implementing surface plasmon resonance biosensors in drug discovery. Pharmaceutical Science and Technology Today 3, 310-317.
Namba, K. and Vonderviszt, F. (1997) Molecular architecture of bacterial flagellum. Quart. Rev. Biophys. 30, 1-65.
Namba, K., Yamashita, I. & Vonderviszt, F. (1989) Structure of the core and central channel of bacterial flagella. Nature 342, 648-654.
Nygren P. & Uhlen, M. (1997) Scaffolds for engineering novel binding sites in proteins. Curr. Op. Struct. Biol. 7, 463-469.
Plückthun, A., Hanes, J., Jermutus L., (2001) Polysome display in the absence of functional ssrA-RNA, U.S. Pat. No. 6,589,741
Rich, R. L. and Myszka, D. G. (2000) Advances in surface plasmon resonance biosensor analysis. Curr. Op. Biotechnol. 11, 54-61.
Roberts R. W., Szostak J. W. Roberts et al., (1997) RNA-peptide fusions for the in vitro selection of peptides and proteins. Proc Natl Acad Sci USA. 94(23) 12297-12302
Samatey, F. A., Imada, K., Nagashima, S., Vonderviszt, F., Kumasaka, T., Yamamoto, M. and Namba, K. 2001) Structure of the bacterial flagellar protofilament and implications for a switch for supercoiling. Nature 410, 331-337.
Sambrook D., Russel D. W., Molecular Cloning A Laboratory Manual, (2001) 3^{rd} edition, Cold Spring Harbor Laboratory Press, Cold Spring Harbor N.Y.
Schoenhals, G, Whitefield, C., (1993) Comparative analysis of flagellin sequences from Escherichia coli strains possessing serologically distinct flagellar filaments with a shared complex surface pattern. J. Bacteriol, 175(17), 5395
Skerra, A. (2001) Anticalins: a new class of engineered ligand-binding proteins with antibody-like properties. Rev. Mol. Biotech. 74, 257-275.
Smith, N. & Selander, R. K. (1990) Sequence invariance of the antigen-coding central region of the phase-I flagellar filament gene (fliC) among strains of Salmonella typhimurium. J. Bacteriol. 172, 603-609.
Stemmer, W. P. (1994) Rapid evolution of a protein in vitro by DNA shuffling. Nature, 370(6488) 389-391
Stocker B. A. and Newton S. M. (1994) Immune responses to epitopes inserted in Salmonella flagellin, Int. Rev. Immunol 11(2), 167-178.
Takahashi, T. T., Austin, R. J. and Roberts, R. W. (2003) mRNA display: ligand discovery, interaction analysis and beyond. Trends Biochem. Sci. 28, 159-165.
Tao, H. and Cornish, V. W. (2002) Milestones in directed enzyme evolution. Curr. Op. Chem. Biol. 6, 858-864.

Vinekas B., Ge L., Pluckthun A., Schneider K. C., Wellnhofer G., Moroney S. E. (1994) Trinucleotide phosphoramidites: ideal reagents for the synthesis of mixed oligonucleotides for random mutagenesis. Nucleic Acids Res. 22(25) 5600-7.
Vonderviszt, F., Aizawa, S.-i. and Namba, K. (1991) Role of the disordered terminal regions of flagellin in filament formation and stability. J. Mol. Biol. 221, 1461-1474.
Vonderviszt, F., Kanto, S., Aizawa, S. I. and Namba, K. (1989) Terminal regions of flagellin are disordered in solution. J. Mol. Biol. 209, 127-133.
Wakabayashi, K., Hotani, H., Asakura, S. (1969) Polymerization of flagellin in the presence of high concentration of salts. Biochim. Biophys. Acta 175, 195-203.
Wei, L. N. & Joys, T. M. (1985) Covalent structure of three phase-I flagellar filament proteins of Salmonella. J. Mol. Biol. 186, 791-803.
Westerlund-Wikström, B. (2000) Peptide display on bacterial flagella: Principles and applications Int. J. Med. Microbiol. 290, 223-230
Wu, Y. J., Newton, S., Judd, A., Stocker B., Robinson, W. S., (1989) Proc. Natl. Acad. Sci. USA, 86 4726-4730
Wybourne, M. N., Keana J. F. W., Cai S. X., Yan. M., Wu J. (1995) Sensors employing interference of electromagnetic waves passing through waveguides having functionalized surfaces. U.S. Pat. No. 5,465,151
Xu, L. at al. (2002) Directed evolution of high-affinity antibody mimics using m-RNA display. Chemistry & Biology 9, 933-942.
Yamashita, I., Vonderviszt, F., Noguchi, T. and Namba, K. (1991) Preparing well-oriented sols of straight bacterial flagellar filaments for X-ray fiber diffraction. J. Mol. Biol. 217, 293-302.
Yonekura, K., Maki, S., Morgan, D. G., DeRosier, D. J., Vonderviszt, F., Imada, K. and Namba, K. (2000). The bacterial flagellar cap as the rotary promoter of flagellin self-assembly. Science 290, 2148-2152.
Yonekura, K., Maki-Yonekara, S. and Namba, K. (2003) Complete atomic model of the bacterial flagellar filament by electron cryomicroscopy. Nature 424, 643-650.
Zaccolo, M., Williams, D. M., Brown, D. M., Gherardi, E. (1996) J. Mol. Biol. 255, 589-603
Zhu, H. and Snyder, M. (2003) Protein chip technology. Curr. Op. Chem. Biol. 7, 55-63.

1-46. (canceled)

47. A filamentous receptor structure comprising a reconstituted flagellar filament said filament comprising at least one or at least 10 or at least 20 or at least 50, artificially modified flagellin as a receptor subunit, wherein in the receptor subunit:
the variable region, preferably the D3 domain, is modified relative to the variable region of a wild type flagellin, and a binding site capable of recognizing and binding a target substance is constructed thereon, and
the terminal regions responsible for polymerization are unmodified, or modified only to such an extent that they preserve their polymerization ability.

48. The filamentous receptor structure of claim 47, wherein the target substance differs from immunoglobulins and immunoglobulin fragments and from receptor proteins.

49. The filamentous receptor structure of claim 47, wherein said filament comprises at least two types of flagellin subunits among which there is at least one artificially modified receptor subunit as defined in claim 47.

50. The filamentous receptor structure of claim 49, wherein
the at least two types of flagellin subunits are any of the following:
a) at least two types of receptor subunits, and/or
b) at least one type of receptor subunit and a subunit with a structure essentially similar to that of the wild type flagellin, and/or
c) a receptor subunit and separately a detecting subunit that is capable of changing at least one of its detectable properties as a result of one or more events of binding of the target substance, and/or
d) any combination of the a), b) and c).

51. The filamentous receptor structure of claim 50, which is stabilized against depolymerization by disulphide bridges formed between adjacent subunits.

52. The filamentous receptor structure of claim 47, which is attached to a carrier.

53. The filamentous receptor structure of claim 47, wherein the receptor subunit is selected from
a flagellin receptor subunit comprising an ion binding site or a metal ion binding site,
a flagellin receptor subunit obtainable by directed evolution, and comprising one or more than one modified peptide segments forming a binding site capable of recognizing and binding the target substance,
a flagellin receptor subunit comprising a modified D3 domain compared with a D3 domain of a wild type flagellin of a given bacterium species or strain, wherein the D3 domain comprises one or more modified peptide segments inserted into one or more hypervariable region(s) and/or into one or more surface loop regions of the D3 domain and/or the peptide segment(s) replace(s), partly or entirely, one or more of said regions, and the peptide segments or a combination of the peptide segments form a binding site capable of recognizing and binding the target substance, wherein the inserted peptide segment is not a thioredoxine-like protein or thioredoxin,
flagellin receptor subunit containing a moiety carrying a binding site and conjugated with the D3 domain, where the molecule or the moiety is preferably any of the following: a complexing group, e.g. a group capable of binding ions, etc., a protein or protein domain having receptor properties and/or a binding site or binding domain of a protein.

54. A method for use of an isolated, reconstituted flagellar filament as a receptor, said filament comprising at least one modified flagellin, preferably a multiplicity of modified flagellins
the modified flagellin is modified in its variable region relative to the variable region of the wild type flagellin, to comprise a binding site capable of recognizing and binding a target substance, and
at least the terminal regions of the flagellin, responsible for the polymerization, are unmodified or modified only to an extent that the said terminal regions preserve their polymerization ability,
said method comprising the steps of,
contacting the studied target substance with the isolated flagellar filament under conditions allowing the binding to the modified flagellin,
detecting the binding of the target molecule to the modified flagellin subunits of the isolated flagellar filament,
wherein the target molecule differs from immunoglobulins or immunoglobulin fragments and differs from receptor proteins.

55. The method of claim 54, wherein said filament comprises at least two types of flagellin subunits selected from
a) at least two types of receptor subunits, and/or
b) at least one type of receptor subunit and a subunit with a structure essentially similar to that of the wild type flagellin, and/or
c) a receptor subunit and separately a detecting subunit that is capable of changing at least one of its detectable properties as a result of one or more events of binding of the target substance, and/or
d) any combination of the a), b) and c).

56. The method of claim 54 wherein the receptor subunit is selected from
a flagellin receptor subunit comprising an ion binding site or a metal ion binding site,
a flagellin receptor subunit obtainable by directed evolution, and comprising one or more than one modified peptide segments forming a binding site capable of recognizing and binding the target substance,
a flagellin receptor subunit comprising a modified D3 domain compared with a D3 domain of a wild type flagellin of a given bacterium species or strain, wherein the D3 domain comprises one or more modified peptide segments inserted into one or more hypervariable region(s) and/or into one or more surface loop regions of the D3 domain and/or the peptide segment(s) replace(s), partly or entirely, one or more of said regions, and the peptide segments or a combination of the peptide segments form a binding site capable of recognizing and binding the target substance, wherein the inserted peptide segment is not a thioredoxine-like protein or thioredoxin,
flagellin receptor subunit containing a moiety carrying a binding site and conjugated with the D3 domain, where the molecule or the moiety is preferably any of the following: a complexing group, e.g. a group capable of binding ions, etc., a protein or protein domain having receptor properties and/or a binding site or binding domain of a protein.
